(19) United States
(12) Patent Application Publication
(10) Pub. No.: US 2004/0180022 A1
(43) Pub. Date: Sep. 16, 2004

(54) Solubilizers for aqueous detergent compositions, containing an oily substance

(76) Inventor: Selcuk Denzligil, Istanbul (TR)

Correspondence Address: COGNIS CORPORATION, PATENT DEPARTMENT, 300 BROOKSIDE AVENUE, AMBLER, PA, 19002, US

(21) Appl. No.: 10/762,386

(22) Filed: Jan. 22, 2004

(30) Foreign Application Priority Data
Jan. 22, 2003 (EP) EP 03001428.6

(51) Int. Cl. A61K 7/06; A61K 7/11; A61K 7/075; A61K 7/08

(52) U.S. Cl. 424/70.13; 424/70.24; 424/70.31

(57) ABSTRACT

A cleaning composition comprising: (a) an oily substance; and (b) a solubilizer containing: (i) a first surfactant selected from the group consisting of a fatty alcohol ethoxylate corresponding to formula (I): R¹—(C₂H₄O)ₙ—H, wherein R¹ is a linear or branched, saturated or unsaturated alkyl moiety having from about 6 to 22 carbon atoms, and n is an integer from 1 to about 10, a fatty alcohol alkoxylate corresponding to formula (II) R²—(C₂H₄O)ₙ—(C₃H₆—O)ₘ—H, wherein R² is a linear or branched, saturated or unsaturated alkyl moiety having from about 6 to 22 carbon atoms, and n and m are, independently from one another, an integer from 1 to about 10, and mixtures thereof; (ii) an alkyl and/or alkenyl oligoglycoside corresponding to formula (III): R³O—[G]ₚ wherein 3 is a branched and unbranched alkyl and/or alkenyl radical having from about 4 to 22 carbon atoms, G is a sugar radical having 5 or 6 carbon atoms, and p is a number from 1 to 10; (iii) a fatty alcohol ether sulfate corresponding to formula (IV): R⁴—(C₂H₄O)ₙ—SO₃⁻K⁺ wherein le is a linear or branched, saturated or unsaturated alkyl moiety having from about 6 to 22 carbon atoms, n is a number from 1 to about 10, and K⁺ represents a cation; and (iv) optionally an acid component.

### BACKGROUND OF THE INVENTION

The present invention concerns to a solubilizer system, containing various nonionic surfactants in combination with alkyl ether sulfates and the use of such systems in cleansing and laundry detergent compositions, containing oily substances, for instance perfumes.

Lipophilic substances, like vitamins or perfumes are often used in laundry and cleansing detergent Air freshener compositions. However, to incorporate such non-water soluble substances into the compositions it is common to add so-called solubilizers. Such compounds—which often have an intermediate HLB-value, are able to build a bridge between the polar solvent and the non-polar active ingredient. Such solubilizers are well-known in the art. Reference is made to WO 01/90245, where a solubilizer system for cosmetic and pharmaceutical compositions is disclosed, containing alkoxylated fatty alcohols in combination with ethoxylated triglycerols as effective solubilizer composition. However, the use of those systems cannot be transferred directly into laundry detergent composition, because of the different ingredients and pH-values in those compositions.

The problem, addressed by the present invention was, to find effective solubilizer systems, which can be used in laundry detergent containing formulations, to incorporate oily substances, preferred perfumes. Additionally, the solubilizer should be easy to formulate into the final compositions, and should not effect the stability of the whole composition, even under severe temperature conditions.

It was found, that the combination of selected non-ionic surfactants with alkyl ether sulfates can solve the problem.

### BRIEF SUMMARY OF THE INVENTION

The subject matter of the main claim is therefore as follows:

A solubilizer composition, containing at least,

a) a fatty alcohol ethoxylate according to formula (I) R¹—(C₂H₄O)ₙ—H, where R¹ stands for a linear or branched, saturated or unsaturated alkyl moiety with 6 to 22 C-atoms, and n is m integer from 1 to 10, and/or

b) a fatty alcohol alkoxylate according to formula (II) R²—(C₂H₄O)ₙ(C₃H₆—O)ₘ—H, where R² stands for a linear or branched, saturated or unsaturated alkyl moiety with 6 to 22 C-atoms, and n and in are independently from each other numbers from 1 to 10, and

c) an alkyl and/or alkenyl oligoglycosides which conform to the formula (X) R³O—[G]ₚ in which R³ is a branched and unbranched allyl and/or alkenyl radical having from 4 to 22 carbon atoms, G is a sugar radical having 5 or 6 carbon atoms, and p stands for numbers from 1 to 10, and

d) a fatty alcohol ether sulfate of formula (IV), R⁴—(C₂H₄O)ₙ—SO₃⁻K⁺ where R⁴ represents a linear or branched, saturated or unsaturated alkyl moiety with 6 to 22 C-atoms, n is a number of 1 to 10 and K⁺ represents a cation.

The solubilizer system according to the present invention contains a mixture of three different classes of substances, at least one kind of alkoxylated fatty alcohol like compounds a) and/or b), in combination with an alkyl- or alkenyl(oligo)-glycoside as compound c) and an anionic compound d). It is preferred that the weight ratio of compounds a) and/or b): c): d) is in the range from 1:10:10 to 1:1:1 and preferred in the range from 1:2:2 to 1:5:5.

### DETAILED DESCRIPTION OF THE INVENTION

Compound a)

Fatty alcohol ethoxylates according to formula (I) R¹—(C₂H₄O)ₙ—H, where R¹ stands for a linear or branched, saturated or unsaturated alkyl moiety with 6 to 22 C-atoms, and n is an integer from 1 to 10, are well kwon substances, which can be prepared by known methods of organic chemistry. In a preferred embodiment of the present invention a fatty alcohol ethoxylate according to formula (I), wherein R¹ stands for a linear, unsaturated alkyl chain with 8 to 20 C-atoms, and n is a number from 5 to 10 is used. Of such alcohol ethoxylates, also suitable are those which have a narrowed homolog distribution.

Compound b)

Like compound a) also substances in accordance with formula (II) R²—(C₂H₄—O)ₙ(C₃H₆—O)ₘ—H, where R² stands for a linear or branched, saturated or unsaturated alkyl moiety with 6 to 22 C-atoms, and n and m are independently from each other numbers from 1 to 10, are well known. It is preferred to use compounds b) according to formula (II), where R² stand for an linear, unsaturated alkyl chain with 8 to 20 C-atoms, and n is a number from 1 to 10 and m is a number from 1 to 3.

Compound c)

To prepare the compositions of the invention it is preferred to use alkyl and/or alkenyl oligoglycosides which conform to the formula (III)

R³O—[G]ₚ (III)

in which 3 is a branched and unbranched alkyl and/or alkenyl radical having from 4 to 22 carbon atoms, G is a sugar radical having 5 or 6 carbon atoms, and p stands for numbers from 1 to 10. They are preferably prepared by reacting glucose or dextrose monohydrate and fatty alcohol in the presence of catalysts. In this context they may be obtained by relevant processes of preparative organic chemistry. The alkyl and/or alkenyl oligoglycosides may derive from aldoses and/or ketoses having 5 or 6 carbon atoms, preferably from glucose. The preferred alkyl and/or alkenyl oligoglycosides are therefore alkyl and/or alkenyl oligoglucosides. The index p in the general formula an) indicates the degree of oligomerization (DP), i.e., the distribution of monoglycosides and oligoglycosides, and stands for a number between 1 and 10. While p in a given compound must always be integral and in this case may adopt in particular the values p=1 to 6, p for a particular alkyl oligoglycoside is an analytically determined arithmetic variable which usually represents a fraction. Preference is given to using alkyl and/or alkenyl oligoglycosides having an average degree of oligomerization p of from 1.1 to 3.0. From a performance standpoint, preference is given to alkyl and/or alkenyl oligoglycosides whose degree of oligomerization is less than 1.7 and is in particular between 1.2 and 1.5. The alkyl and/or alkenyl radical R1 may derive from primary alcohols having from 4 to 11, preferably from 8 to 10, carbon atoms. Typical examples are butanol, caproyl alcohol, caprylyl alcohol, capryl alcohol, and undecyl alcohol, and their technical-grade mixtures, as obtained, for example, in the hydrogenation of technical-grade fatty acid methyl esters or in the course of the hydrogenation of aldehydes from the Roelen oxo process. Preference is given to alkyl oligoglucosides of chain length C8-C10 (DP=1 to 3), which are obtained as the initial fraction during the distillative separation of technical-grade C8-C18 coconut fatty alcohol and may have an impurities fraction of less than 6% by weight of C12 alcohol, and also alkyl oligoglucosides based on technical-grade C9/11 oxo alcohols (DPP=1 to 3). The alkyl and/or alkenyl radical R³ may also derive from primary alcohols having from 12 to 22, preferably from 12 to 18, carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, and their technical-grade mixtures, which may be obtained as described above. Preference is given to alkyl oligoglucosides based on hydrogenated C12/14 cocoyl alcohol with a DP of from 1 to 3.

Compound d

Alkyl ether sulfates (“ether sulfates”) constitute known anionic surfactants which are prepared industrially by SO₃ or chlorosulfonic acid (CSA) sulfation of fatty alcohol or oxo alcohol polyglycol ethers and subsequent neutralization. Ether sulfates suitable in the context of the invention are those which conform to the formula (IV)

R⁴O—(CH₂CH₂O)nSO₃⁻K⁺ (IV)

in which R⁴ is a linear or branched alkyl and/or alkenyl radical having from 6 to 22 carbon atoms, a stands for numbers from 1 to 10, and K⁺ is preferred an alkali metal and/or alkaline earth metal, ammonium, alkylammonium, alkanolammonium or glucammonium, Typical examples are the sulfates of adducts of on average from 1 to 10 and in particular from 2 to 5 mol of ethylene oxide with caproyl alcohol, caprylyl alcohol, 2-ethylhexyl alcohol, capryl alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, and brassidyl alcohol, and also their technical-grade mixtures in the form of their sodium and/or magnesium salts. The ether sulfates may have either a conventional or a narrowed homolog distribution. Particularly preferred is the use of ether sulfates based on adducts of on average from 2 to 3 mol of ethylene oxide with technical-grade C12/14 and/or C12/18 coconut fatty alcohol fractions in the form of their sodium and/or magnesium salts.

It is preferred to use those solubilizer systems, which contain compounds a) and/or b) in amounts from 1 to 20% by weight, preferably from 2 to 15% by weight, and especially preferred from 5 to 10% by weight, according to the whole composition. Compound c) is preferably present in in amounts from 10 to 60% by weight, preferred from 15 to 50% by weight and especially preferred from 25 to 50% by weight, according to the whole composition, and at least, compound d) is present in amounts from 10 to 80% by weight, is preferred from 25 to 60% by weight and especially preferred from 30 to 55% by weight, according to the whole composition.

The solubilizing systems according to the present invention are preferred useful for the formulation of aqueous detergent compositions, containing an oily substance, preferably an perfume. As oily substance those compounds are meant, which are liquid at room temperature (21° C.) but not water soluble or miscible with water at 21° C. As perfume oils and/or fragrances it is possible to use certain odorant compounds, examples being the synthetic products of the ester, ether, aldehyde, ketone, alcohol and hydrocarbon type. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenylglycinate, allyl cyclohexylpropionate, styrallyl propionate, and benzyl salicylate. The ethers include, for example, benzyl ethyl ether; the aldehydes include, for example, the linear alkanals having 8-18 carbon atoms, citral, citronellal citronellyloxyacetaldehyde, cylamen aldehyde, hydroxycitronellal, lilial, and bourgeonal; the ketones include, for example, the ionones, isomethylionone and methyl cedryl ketone; the alcohols include anethole, citronellol, eugenol, geraniol, linalool, phenylethyl alcohol and terpineol; and the hydrocarbons include primarily the terpenes such as limonene and pinene. Preference, however, is given to the use of mixtures of different odorants, which together produce an appealing fragrance note. Such perfume oils may also contain natural odorant mixtures, such as are obtainable from plant sources, examples being pine oil, citrus oil, jasmine oil, patchouli oil, rose oil or ylang-ylang oil. Likewise suitable are muscatel, sage oil, camomile oil, clove oil, balm oil, mint oil, cinnamon leaf oil, lime blossom oil, juniperberry oil, vetiver oil, olibanum oil, galbanum oil, and labdanum oil, and also orange blossom oil, nerol oil, orangepeel oil, and sandalwood oil.

The solubilizing system according to the invention may further comprise some other additives. Especially preferred is the co-use of acid substances, especially of hydroxyl carboxylic acids. A preferred acid is citric acid. These acid compounds arm useful to stabilise the solubilizing system, but are not essential. If present, the acids are used in amounts from 0.1 to 3% by weight, according to the whole solubilizing composition.

The use of the solubilizing system according to the present invention is preferred in detergent compositions, containing higher amounts of oily substances, especially of fragrances, like toilet cleaners or air fresheners. These compositions contain, besides the solubilizing system, other common ingredients, like nonionic, anionic and/or cationic surfactants, builders, co-builders, inorganic salts, defoamers, optical brighteners, polymers, greying inhibitors, dyes, enzymes, solvents, bleaches and bleach activators, and, preferably thickeners. The latter ones are often used in toilet cleaners or similar home care product.

Fully formulated compositions in accordance with the present invention can preferably contain from 1 to 30% by weight of a solubilizing system from 1 to 40% by weight of surfactants and from 10 to 90% by weight of water. Other ingredients may be present in amounts from 0.1 to 25% by weight.

The use of the solubilizing systems in aqueous detergent compositions, leads to clear stable compositions with appropriate viscosity behaviour. If a gel consistence is needed, additional thickeners can be incorporated, preferred compounds in this respect are hydroxyl cellulose, xanthan gum or polyacrylates. Such thickened systems may show viscosity's, measured at 21° C. up to 20.000 mPas.

### EXAMPLES

Two different solubilizer compositions were prepared and tested in various toilet cleaner compositions.

| Component | Solubilizer 1% | Solubilizer 2% |
|---|---|---|
| C12-C14 Alkyl ether sulfate + 2 EO, | 49.05 | 49.05 |
| sodium salt | | |
| C8-C10 APG | 41.52 | 41.52 |
| C12-C18 Fatty alcohol + 7 EO | 9.13 | — |
| C12 Fatty alcohol + 1 PO + 9 EO | — | 9.13 |
| Citric acid | 0.3 | 0.3 |

Typical compositions are for toilet cleaner without viscosity:

| | | |
|---|---|---|
| 1) | Water | rest |
| | Solubilizer 1 | 16.5% |
| | Fragrance A | 4% |
| | 5-bromo-5nitro-1,3 dioxane (Bronidox L) | 0.1% |
| | A clear solution is obtained. | |
| 1a) | Water | rest |
| | Solubilizer 2 | 16% |
| | Fragrance A | 4% |
| | 5-bromo-5nitro-1,3 dioxane (Bronidox L) | 0.1% |
| | A clear solution is obtained. | |
| 2) | Water | rest |
| | Solubilizer 1 | 6% |
| | Fragrance B | 2% |
| | 5-bromo-5nitro-1,3 dioxane (Bronidox L) | 0.1% |
| | A clear solution is obtained. | |
| 2a) | Water | rest |
| | Eumulgin HRE 40 | 12% |
| | Fragrance B | 2% |
| | 5-bromo-5nitro-1,3 dioxane (Bronidox L) | 0.1% |
| | A clear solution is obtained. | |

Furthermore, tests were conducted to show the effect of the solubilizing system of the invention in dissolving oily substances in water in comparison to products, known in the art. For this test two fragrances, Citrone terpene and Fragrance A (each 1 g per litre) were dissolved in an aqueous system, using various amounts of a solubilizing system for a complete dissolution of the fragrance. The temperature was 21° C. In the following table the amount of solubilizing systems used is listed.

| Solubilizer | Citrone terpene | Fragrance A |
|---|---|---|
| Solubilizer 1 | 2.3 g | 2.3 g |
| Solubilizer 2 | 2.5 g | 2.5 g |
| C12-C18 Fatty alcohol + 7 EO | 12.0 g | 14.0 g |
| Blend of C12-C18-Mono-Di- | 8.2 g | 6.0 g |
| Triglycerides | | |
| Polyoxyethylen(20)sorbitanmonolaurat | 8.0 g | 6.5 g |

It could be proofed that the solubilizing system according to the invention is more effective than solubilizers known from prior art.

1: A composition comprising:
(a) an oily substance; and
(b) a solubilizer containing:
(i) a first surfactant selected from the group consisting of a fatty alcohol ethoxylate corresponding to formula (I):
R¹—(C₂H₄O)ₙ—H, wherein R¹ is a linear or branched, saturated or unsaturated alkyl moiety having from about 6 to 22 carbon atoms, and n is an integer from 1 to about 10, a fatty alcohol alkoxylate corresponding to formula (II):
R²—(C₂H₄O)ₙ—(C₃H₆—O)ₘ—H, wherein R² is a linear or branched, saturated or unsaturated alkyl moiety having from about 6 to 22 carbon atoms, and n and m arm, independently from one another, an integer from 1 to about 10, and mixtures thereof;
(ii) an alkyl and/or alkenyl oligoglycoside corresponding to formula (III):
R³O—[G]ₚ wherein R³ is a branched and unbranched alkyl and/or alkenyl radical having from about 4 to 22 carbon atoms, G is a sugar radical having 5 or 6 carbon atoms, and p is a number from 1 to 10;
(iii) a fatty alcohol ether sulfate corresponding to formula (IV):
R⁴—(C₂H₄O)ₙ—SO₃⁻K⁺ wherein R⁴ is a linear or branched, saturated or unsaturated alkyl moiety having from about 6 to 22 carbon atoms, n is a number from 1 to about 10, and K⁺ represents a cation; and
(iv) optionally, an acid component.

2: The composition of claim 1 wherein (i) is present in the solubilizer in an amount of from about 1 to 20% by weight, based on the weight of the solubilizer.

3: The composition of claim 1 wherein (i) is present in the solubilizer in an amount of from about 2 to 15% by weight, based on the weight of the solubilizer.

4: The composition of claim 1 wherein (i) is present in the solubilizer in an amount of from about 5 to 10% by weight, based on the weight of the solubilizer.

5: The composition of claim 1 wherein (ii) is present in the solubilizer in an amount of from about 10 to 60% by weight, based on the weight of the solubilizer.

6: The composition of claim 1 wherein (ii) is present in the solubilize in an amount of from about 25 to 50% by weight, based on the weight of the solubilizer.

7: The composition of claim 1 wherein (iii) is present in the solubilizer in an amount of from about 10 to 80% by weight, based on the weight of the solubilizer.

8: The composition of claim 1 wherein (iii) is present in the solubilizer in an amount of from about 25 to 60% by weight, based on the weight of the solubilizer.

9: The composition of claim 1 wherein (iii) is present in the solubilizer in an amount of from about 30 to 55% by weight, based on the weight of the solubilizer.

10: The composition of claim 1 wherein (iv) is present in the solubilizer in an amount of from about 0.1 to 3% by weight, based on the weight of the solubilizer.

11: The composition of claim 1 wherein the solubilizer is present in the composition in an amount of from about 1 to 30% by weight, based on the weight of the composition.

12: A process for solubilizing an oily substance comprising:
(a) providing an oily substance;
(b) providing a solubilizer containing:
(i) a first surfactant selected from the group consisting of a fatty alcohol ethoxylate corresponding to formula (I):
R¹—(C₂H₄O)ₙ—H, wherein R¹ is a linear or branched, saturated or unsaturated alkyl moiety having from about 6 to 22 carbon atoms, and n is an integer from 1 to about 10, a fatty alcohol alkoxylate corresponding to formula (II);
R²—(C₂H₄O)ₙ—(C₃H₆—O)ₘ—H, wherein R² is a linear or branched saturated or unsaturated alkyl moiety having from about 6 to 22 carbon atoms, and n and m are, independently from one another, an integer from 1 to about 10, and mixtures thereof;
(ii) an alkyl and/or alkenyl oligoglycoside corresponding to formula (III):
R³O—[G]ₚ wherein R³ is a branched and unbranched alkyl and/or alkenyl radical having from about 4 to 22 carbon atoms, G is a sugar radical having 5 or 6 carbon atoms, and p is a number from 1 to 10;
(iii) a fatty alcohol ether sulfate corresponding to formula (IV):
R⁴—(C₂H₄O)ₙ—SO₃⁻K⁺ wherein R⁴ is a linear or branched, saturated or unsaturated alkyl moiety having from about 6 to 22 carbon atoms, n is a number from 1 to about 10, and K⁺ represents a cation; and
(iv) optionally, an acid component; and
(c) combining (a) and (b).

13: The process of claim 12 wherein (i) is present in the solubilizer in an amount of from about 1 to 20% by weight, based on the weight of the solubilizer.

14: The process of claim 12 wherein (i) is present in the solubilizer in an amount of from about 5 to 10% by weight, based on the weight of the solubilizer.

15: The process of claim 12 wherein (ii) is present in the solubilizer in an amount of from about 10 to 60% by weight, based on the weight of the solubilizer.

16: The process of claim 12 wherein (ii) is present in the solubilizer in an amount of from about 25 to 50% by weight, based on the weight of the solubilizer.

17; The process of claim 12 wherein (iii) is present in the solubilizer in an amount of from about 10 to 80% by weight, based on the weight of the solubilizer.

18: The process of claim 12 wherein (iii) is present in the solubilizer in an amount of from about 30 to 55% by weight, based on the weight of the solubilizer.

19: The process of claim 12 wherein (iv) is present in the solubilizer in an amount of from about 0.1 to 3% by weight, based on the weight of the solubilizer.

20: The process of claim 12 wherein the solubilizer is present in the composition in an amount of from about 1 to 30% by weight, based on the weight of the composition.
